(19) United States
(12) Patent Application Publication
(10) Pub. No.: US 2022/0361959 A1
(43) Pub. Date: Nov. 17, 2022

(54) System and Method for Computation of Coordinate System Transformations

(71) Applicant: Universitat Bern, Bern (CH)

(72) Inventors: Stefan Weber, Herrenschwanden (CH); Andreas Raabe, Muri (CH)

(21) Appl. No.: 17/767,036

(22) PCT Filed: Oct. 6, 2020
(86) PCT No.: PCT/EP2020/078017

§ 371 Date: Apr. 6, 2022

(30) Foreign Application Priority Data
Oct. 6, 2019 (EP) 19201585.7

(51) Int. Cl. A61B 34/20; A61B 34/30

(52) U.S. Cl. CPC A61B 34/20; A61B 34/30; A61B 2090/3966

(57) ABSTRACT

The present invention relates to a medical system (100) for determining a coordinate transformation between a coordinate system (INN₁) of an internal structure (I₁) inside a physical object (1) and a coordinate system (IMA) of a 3D image or model thereof.

The present invention relates to a medical system and to a corresponding method, and particularly to medical tomographic imaging and surgical arts. It finds particular application in conjunction with image-guided surgery (IGS) and robotic surgery but is also amenable to other applications.

Medical tomographic imaging is valuable for obtaining accurate models of a patient's internal anatomy and/or pathology in a non-invasive manner. Prior to a procedure, a tomographic image data set of anatomy of interest may be generated by CT or Cone Beam scanners, magnetic resonance imaging (MRI) scanners, gamma cameras, and other medical diagnostic imaging equipment. Typically, these imaging modalities provide structural detail with a sub-millimetric resolution. Reconstructed images of the anatomy can be used throughout surgical or interventional procedures to aid in navigating through and/or around various anatomical structures.

Generally, IGS systems include a computer and systems for spatial and temporal tracking of instruments and aspects of the patient's anatomy by means of optical tracking, magnetic tracking, time-of-flight tracking or other means. Upon co-registration of a tomographic medical image data set and the available pose data (relative pose between the instrument's pose and the target structure's pose), the corresponding pose of a virtual instrument within the medical image data set can be computed and displayed accordingly. Moreover, surgical robots can be guided via open- or closed loop control mechanisms using available tracking information.

Various stereotactic IGS procedures have been developed taking advantage of the tomographic image data of the patient, including but not limited to needle biopsies, shunt placements, tumour ablations, craniotomies and cochlear implantations.

Another IGS procedure is spinal fusion surgery, including screw placement and fixation, fracture decompression, and spinal tumor removal. Specifically, during spinal screw fixation procedures, screw holes are created in spinal vertebra into which a screw is threaded. Surgeons rely on IGS or fluoroscopic guidance for optimal placement of hole and screw. Unaided, or conducted using current guidance modalities, this approach can lead to less than optimal placement of screws which in turn may injure nerves, blood vessels, or the spinal cord.

Nevertheless, the use of IGS is associated to inaccuracies in the alignment of the subject's real world anatomy with its corresponding model in image space. In the actual surgical procedure, a structure's position and orientation may change as a result of physical manipulation and applied forces through instruments resulting in a geometric error relative to the preoperatively acquired image. This error will eventually result in an inaccurate guidance of an instrument or tool which in turn may lead to a surgical complication or suboptimal surgical result. The geometric error decreases when distances are small, (for example smaller than 10 cm) and the same solid and rigid segment (for example one vertebra, the skull, one bone without joint, etc.) is used for registration and as a target for IGS. The geometric error increases when distances increase (for example larger than 20 cm) and more than one segment is involved, and the segments are flexibly attached to each other (two or more vertebrae, bones with joints, etc.), and the segment used for registration is different from the segment used as a target for IGS.

In many current applications, only one segment (such as the sacrum) is tracked by an available tracking system, and another segment is targeted for IGS (the segment where the surgical procedure is performed) resulting in ever increasing spatial errors for segments farther afield from the tracked segment. In cases where the relative spatial relationships of various anatomical features of interest have shifted or are otherwise changed in comparison to when the image was obtained, screw misplacement is to be expected.

Accordingly, a geometric transform registers a subject's specific target vertebra (being tracked in space and time) with a corresponding image (in image space). Subject to dynamic deformation caused by manipulation, breathing etc. and by static displacement created by prior manipulation, a single transformation cannot accurately map any other vertebra to the corresponding image in image space. Subsequently, a given transformation can only map the surgical tools pose relative to the target vertebra accurately. Any other vertebra cannot be navigated using the given transformation. As a result, instrument guidance will result in inaccurate representation of the relative spatial relationship between the surgical tool and the nearby anatomy.

To date, no system is available to allow for tracking of more than one non-rigid anatomical structure (i.e. the various segments of the spine) inside another structure (the subjects body). Furthermore, no method is available to derive a geometric transformation by transfer of coordinate systems from features outside the body to features inside the body (also called internal structures herein). Likewise, no system is available for splitting up a coordinate system that is applied to a several-segment-system (e.g. more than two vertebrae of the spine) into several sub-coordinate systems and to hand-over these sub-coordinate systems one-by-one to specific one-segment-units (i.e. only one vertebra), without loss of accuracy and without additional imaging.

In the state of the art, various approaches are described to register internal structures to a model data set, including methods registering a three dimensional model with various imaging modalities such as
a. ultrasound (W02012177470, DE102011106812),
b. fluoroscopy (US2011270072, EP3326564);
c. thermographic imaging (US2012281898);
d. optical surface scanning (WO2016015760, WO2015074158, WO2014094811);
e. video based imaging (EP1657678, mono-focal DE102012208389);

or via using non-imaging approaches such as
f. generic image information (ie. atlas) based approaches (EP2912630);
g. imaging of additional reference structures (U.S. Pat. No. 6,674,916);
h. visual alignment by human and through using generic graphical user interfaces (US2008300477) and/or augmented reality based systems (WO2018171880);

In WO2011063840, fiducials attached to an object are identified in the model of an object and spatial configuration of the fiducials within the model (via image analysis) and within the object (via tracking) is used to determine spatial shifts of the fiducials.

Fiducials (artificial landmarks) can be automatically determined in an image volume using prior knowledge about geometric (size, shape) and physical (i.e. density) properties (EP0732899 and U.S. Pat. No. 5,769,789). Moreover, system and methods have been disclosed for identifying points on spatial bodies by comparing its spatial information relative to available data from corresponding points in a database (EP0927403). Accordingly, stereotactic surgical procedures can be performed through navigation based upon the relative position of multiple fixed reference points (e.g., fiducials) placed on a patient's anatomy. (WO2018/191057).

Additionally, moving objects can be tracked in space and time by tracking fiducials that are rigidly attached to the body by use of repeated scanning of the object via CT scanner (EP2070478). Also, systems have been disclosed for tracking dynamic reference frames using trackable markers, some of the markers being movable in 3D space. This has been applied to fiducial markers in spinal surgery. (US2019/0209080). Additionally, algorithmic solutions can be used to track structures by means of image analysis in three dimensional image data sets (WO2016206743).

Inventions have been disclosed for registering between a robotic coordinate system and the image data set two positional coordinates spaced apart along a target object (bone) and a directional vector passing through at least one of the positional coordinates. (WO9836371). Similarly, inventions have been disclosed to optimize the tracking of end-effectors of robotic surgical systems relative to tracking arrays on a patient. (US2017/0348061).

Based on the above needs unmet by the state of the art, it is an objective of the present invention to provide a medical system and a method that both allow a precise and accurate tracking of a variety of a subject's internal structures (e.g. individual segments) and tracking/registering that pose information relative to an image coordinate system.

This problem is solved by a medical system having the features of claim 1 as well as by a method having the features of claim 14.

Preferred embodiments of these aspects of the present invention are stated in the corresponding sub-claims and are described in the following.

According to claim 1, a medical system for determining a coordinate transformation between a coordinate system of an internal structure inside a physical object and a coordinate system of a 3D image or model thereof is disclosed, wherein the medical system comprises:
a plurality of surface fiducial markers that are configured to be attached to an outer surface of a physical object, particularly in arbitrary spatial configurations with respect to each other (e.g. a body of a person or a portion of a body of a person), with any geometric arrangement being possible
at least one adapter that is configured to be attached to an internal structure (e.g. a spinal vertebra) of the physical object, wherein the system can comprise at least one structure fiducial marker that is configured to be releasably connected to the at least one adapter (e.g. for tracking of the internal structure during surgery),
a medical imaging unit, for example a computed tomography (CT) scanner or a magnetic resonance imaging (MRI) device, configured to generate a 3D image of said physical object and the surface fiducial markers attached to said outer surface,
a processing unit configured for measuring (e.g. manually or automatically) each surface fiducial marker's pose within the 3D image and relative to an image coordinate system (IMA) of the 3D image and to compute a coordinate system of the surface fiducials (SUR) from the positions of the surface fiducials as well as first coordinate transformation (^{SUR}T_{IMA}) from the image coordinate system to the coordinate system (SUR) of the surface fiducial markers;
a measuring unit (for example a stereotactic camera) for measuring the poses of the surface fiducial markers with respect to a coordinate system (WOR) of the measuring unit when the respective surface fiducial marker is attached to said outer surface,
wherein the processing unit is further configured to compute a second coordinate transformation (^{IMA}T_{WOR}) from the coordinate system (WOR) of the measuring unit into the image coordinate system (IMA) via the coordinate system of the surface fiducial markers (SUR), thereby allowing to reference points on said outer surface to points within the 3D image,
wherein the medical system is configured to measure the pose of the at least one adapter relative to the surface fiducial markers when the at least one adapter is attached to an internal structure of the physical object, and wherein the processing unit is configured to compute a third coordinate transformation ^{INN1}T_{SUR} from the coordinate system (SUR) of the surface fiducials into the coordinate system (INN₁) of the internal structure, and wherein the processing unit is configured to combine the second coordinate transformation ^{IMA}T_{WOR} with the first coordinate transformation (^{SUR}T_{IMA}) and with the third coordinate transformation (^{INN1}T_{SUR}) to create a coordinate transformation (^{INN1}T_{WOR}) from the coordinate system (WOR) of the measuring unit into the coordinate system (INN₁) of the internal structure, thereby allowing to measure the pose of the at least one adapter in the coordinate system of the measuring unit.

Particularly, combining the second, first and third coordinate transformations corresponds to the matrix multiplication

^{INN1}T_{SUR}^{SUR}T_{IMA}^{IMA}T_{WOR}=^{INN1}T_{WOR}.

Particularly, the respective coordinate transformation between a first coordinate system x and a second coordinate system X′ can be represented as a 3×3 matrix T having three vertical columns and three horizontal rows in a well known fashion which expresses how the components of a vector A in the first coordinate system x relate to the components of the same vector A′ in the second coordinate system X′: A′=^{X′}TₓA

The matrix vector multiplication between ^{X′}Tₓ and A yields the components a) of the vector A′ which are determined by multiplication and summation of the entries of the corresponding row of the matrix ^{X′}Tₓ with the components a the vector A:

$${a}_{i}^{′} = {\sum }_{j = 1}^{3} {t}_{ij} {a}_{j}$$

wherein tᵢⱼ are the entries of the matrix ^{X′}Tₓ, wherein i denotes the i-th row and j denotes the j-th column. The inverse matrix (^{X′}Tₓ)⁻¹ is equal to the transpose of ^{X′}Tₓ which is denoted as (^{X′}Tₓ)^{T}. When transposing the matrix, the i-th row, j-th column element of (^{X′}Tₓ)^{T} is the j-th row, i-th column element of ^{X′}Tₓ.

Particularly, as stated above, the processing unit is configured to compute a second coordinate system transformation ^{IMA}T_{WOR} from the coordinate system (WOR) of the measuring unit to the image coordinate system (IMA) via the coordinate system of the surface fiducial markers (SUR). This can be achieved for example, by computing the matrix multiplication between (^{SUR}T_{IMA})⁻¹ and ^{SUR}T_{WOR}:

^{IMA}T_{WOR}=(^{SUR}T_{IMA})^{-1,SUR}T_{WOR},

i.e., by transforming from the coordinate system (WOR) of the measuring unit to the coordinate system (SUR) of the surface fiducial markers and thereafter by transforming from the coordinate system (SUR) of the surface fiducial markers to the image coordinate system (IMA) (the latter transformation corresponds to (^{SUR}T_{IMA})⁻¹).

Due to the specific generation of the respective final coordinate transformation, a subject's specific internal structure (such as a spinal vertebra) can be tracked in space and time relative to a previously acquired image data set. As a result, inaccuracies caused by the above-mentioned sources of translation and error such as dynamic manipulation, respiratory motion and instrument activity can be drastically reduced.

In the framework of the present invention, the notion “position” describes a point or vector in space that comprises three degrees of freedom and can be defined using e.g. three coordinates along linear independent spatial directions (e.g. the coordinate axes x, y, z of a perpendicular right-handed coordinate system).

Furthermore, the notion “pose” describes the spatial position of an extended object and its orientation in space within six degrees of freedom. The pose of an object (such as a fiducial marker) can be defined using e.g. three coordinates along linear independent spatial directions (for example the coordinate axes x, y, z of a perpendicular right-handed coordinate system) as well as rotation angles about these directions/coordinate axes. These angles are often denoted as roll (rotation about x-axis), pitch (rotation about y-axis) and yaw (rotation about z-axis).

Furthermore, according to an embodiment of the medical system, the latter is configured to track said outer surface using for example the surface fiducial markers, wherein particularly the medical system is configured to track the position or the pose of each individual surface fiducial marker, for example by using one of the following techniques: an optical measurement principle, a video-optical measurement principle, an electromagnetic measurement principle, a time-of-flight measurement principle.

Alternatively, or in addition the medical system can be configured to track said outer surface by using one of the following techniques: laser scanning of the outer surface, scanning the outer surface with structured light.

According to an embodiment of the medical system, it is configured to track said outer surface using the surface fiducial markers, wherein particularly the medical system is configured to track the position (3 DOF) or the pose (6 DOF, i.e. position and the orientation) of each individual surface fiducial marker. For this, one of the following can be used: an optical measurement principle, a video-optical measurement principle, an electromagnetic measurement principle, a time-of-flight measurement principle, or any other measurement principle known to the art to be capable of tracking the outer surface using the surface fiducial markers. Alternatively, tracking of the outer surface may also be accomplished by using one of: laser scanning of the outer surface or scanning the outer surface with structured light.

Furthermore, according to an embodiment of the medical system according to the present invention, the medical system comprises different measurement modalities within one coordinate system to allow tracking of surface fiducial markers and structure fiducial markers simultaneously using the different measurement modalities. Furthermore, according to an embodiment of the medical system according to the present invention, the medical system comprises a preferably pose-trackable surgical robotic device configured to generate an access to the internal structure of the physical object/body of the patient and to deliver and particularly to attach the at least one adapter to the internal structure. Particularly, in all embodiments, the internal structure can e.g. be a spinal vertebra of the patient.

The surgical robotic device can be pose tracked with two independent sources of tracking—kinematic tracking and with markers attached to points on the robot.

Furthermore, according to an embodiment of the medical system according to the present invention, the medical system is configured to track several internal structures (e.g. vertebrae) within the same physical object (e.g. body of a patient) independently and to establish and track several geometric transformations between those internal structures and the outer surface of the physical object/body.

Furthermore, according to an embodiment of the medical system according to the present invention, the medical system is configured to relatively track several internal structures against each other and to absolutely track those internal structures against the outer surface simultaneously.

Furthermore, according to an embodiment of the medical system according to the present invention, the at least one adapter comprises a connecting portion that is configured to be releasably connected to at least one structure fiducial marker of the system, and an anchoring portion that is configured to be attached to the internal structure so that an initially registered pose of the adapter or structure fiducial marker relative to the internal structure is reproduced upon re-connection of the released structure fiducial marker to the at least one adapter. Particularly, the connecting portion is connected to the anchoring portion, for example integrally.

Furthermore, according to an embodiment of the medical system according to the present invention, the anchoring portion comprises a thread on an outside of the anchoring portion for anchoring the at least one adapter to the internal structure by screwing the anchoring portion into a bore hole of the internal structure. Particularly, the anchoring portion can be tapered to form a pointed end of the anchoring portion and/or of the thread.

Furthermore, according to an embodiment of the medical system according to the present invention, the connecting portion of the adapter is configured to be arranged on an outside surface of the internal structure when the anchoring portion is anchored to the internal structure, wherein the connecting portion comprises a plurality of image localization features that are integrated into the connecting portion, wherein particularly the respective image localization feature is a radiopaque marker.

Particularly, in an embodiment, the respective image localization features is formed by a cylindrical rod, wherein the rods can be arranged obliquely with respect to one another.

In case the medical system comprises several structural fiducial markers, an adapter is provided for each structural fiducial marker, so that all structural fiducial markers can be attached to an internal structure (e.g. vertebra) via a dedicated adapter.

According to yet another embodiment of the medical system, the image localization feature can be used to update or replace the existing registration transformation ^{INN1}T_{WOR} (for example when the existing transformation has been lost) via an intraoperative imaging method, wherein the previously existing coordinate transformation ^{INN1}T_{WOR} is refined/or replaced by algorithmically locating the internal structure and the localization features of the at least one adapter in the resulting imagery and by computing a subsequent incremental registration transformation ^{INN′}T_{WOR}.

Particularly, for this, the medical system can be configured to compute a coordinate transformation ^{IMA}T_{INN′} from a coordinate system INN′ of the internal structure to an image coordinate system IMA of the intraoperatively obtained image, and by combining this transformation ^{IMA}T_{INN′} with the coordinate transformation ^{IMA}T_{WOR} to achieve said coordinate transformation from the coordinate system WOR of the measuring unit to the coordinate system INN′ of the internal structure:

^{INN′}T_{WOR}=(^{IMA}T_{INN′})^{-1,IMA}T_{WOR}.

Particularly, according to an embodiment of the medical system according to the present invention, the medical system, particularly the processing unit, is configured to compute the coordinate system (SUR) of the surface fiducial markers (Fᵢ) by using a position of a first surface fiducial marker (F₁) as a center of the coordinate system (SUR) of the surface fiducial markers (Fᵢ), wherein the medical system is further configured to use as a first coordinate axis (x) of the coordinate system (SUR) of the surface fiducial markers (Fᵢ) a normalized vector extending from the first surface fiducial marker (F₁) to a second surface fiducial marker (F₂) and as a second coordinate axis (y) a normalized vector extending from the first surface fiducial marker (F₁) to a third surface fiducial marker (F₃) and as a third principal axis (z) the cross product between the first coordinate axis and the second coordinate axis. Further surface fiducial markers may be used to refine accuracy. Other ways of constructing the coordinate system (SUR) of the surface fiducial markers are also conceivable.

Yet another (second) aspect of the present invention relates to a method for determining a coordinate transformation between a coordinate system of an internal structure inside a physical object and an image coordinate system of a 3D image of the internal structure, wherein generating this coordinate transformation comprises the steps of:
(a) computing a first coordinate transformation ^{SUR}T_{IMA} from the image coordinate system (IMA) of the 3D image to a coordinate system (SUR) of the surface fiducial markers attached to an outer side of the physical object (i.e. registration);
(b) computing a coordinate transformation ^{INN1}T_{SUR} from the coordinate system (SUR) of the surface fiducial markers attached to the physical object's outer surface and a coordinate system (INN₁) of an internal structure inside the physical object;
(c) automatically extending the coordinate transformation computed in step (a) by the coordinate transformation computed in step (b) to create a coordinate transformation from the image coordinate system (IMA) of the 3D image to the coordinate system (INN₁) of the internal structure.

Particularly in case the physical object is a body of a living human or animal patient, the method according to the present invention does not comprise any surgical steps. Arranging e.g. the at least one adapter on the internal structure (or several such markers on several internal structures) does not form part of the claimed method.

According to an embodiment of the method according to the present invention, the outer surface is tracked by individually tracking the pose or position of the surface fiducial markers, particularly by means of one of: optical tracking, video-optical tracking, electromagnetic tracking, time-of-flight tracking or any other suitable tracking method known to the art.

Alternatively, instead of such tracking methods, also direct tracking of the outer surface by means of laser scanning or scanning with structured light may be employed according to an embodiment of the method.

Furthermore, according to an embodiment of the method according to the present invention, the surface fiducial markers and the at least one adapter of a structure fiducial marker connected to the at least one adapter are tracked simultaneously using different measurement modalities (see also above).

Particularly, according to an embodiment of the method according to the present invention, several internal structures within the same physical object (e.g. body of a patient) are tracked independently from one another, and wherein a coordinate transformation between each internal structure and the outer surface is established and tracked.

Furthermore, a third aspect of the present invention relates to a computer program comprising instructions which, when the computer program is executed by a computer, cause the computer to carry out the steps of the above-stated method according to the second aspect of the present invention. Further, a fourth aspect of the present invention relates to a computer-readable data carrier having stored thereon the computer program according to the third aspect of the present invention.

Furthermore, according to a fifth aspect of the present invention, an adapter is disclosed, wherein the adapter comprises a connecting portion that is configured to be releasably connected to a fiducial marker, and an anchoring portion that is configured to be attached to an internal structure of a physical object (for example a body of a patient) so that an initially registered pose of the adapter and/or structure fiducial marker relative to the internal structure is reproduced upon re-connection of the released fiducial marker to the adapter. Particularly, the connecting portion is connected to the anchoring portion, wherein the connecting portion can be integrally connected to the anchoring portion. The internal structure can be a bone, for example a vertebra.

Furthermore, according to an embodiment of the adapter, the anchoring portion comprises a thread on an outside of the anchoring portion for anchoring the at least one adapter to an internal structure by screwing the anchoring portion into a bore hole of the internal structure. Particularly, the anchoring portion can be tapered to form a pointed end of the anchoring portion and/or of the thread.

Furthermore, according to an embodiment of the adapter, the connecting portion of the adapter is configured to protrude from on an outside of the internal structure when the anchoring portion is anchored to the internal structure, wherein the connecting portion comprises a plurality of image localization features that are integrated into the connecting portion, wherein particularly the respective image localization feature is a radiopaque marker.

Particularly, in an embodiment of the adapter, the respective image localization feature is formed by a cylindrical rod, wherein the rods can be arranged obliquely with respect to one another.

According to a sixth aspect of the present invention a method is disclosed, wherein the method preferably uses the medical system according to the present invention, and wherein the method comprises the steps of:
generating a 3D image of a physical object using a medical imaging unit, wherein surface fiducial markers are arranged on an outer surface of the physical object,
measuring each surface fiducial marker's pose within the 3D image and relative to an image coordinate system (IMA) of the 3D image, and automatically computing a coordinate system (SUR) of the surface fiducial markers from the positions of the surface fiducial markers, and automatically computing a first coordinate transformation (^{SUR}T_{IMA}) from the image coordinate system (IMA) to the coordinate system (SUR) of the surface fiducial markers;
using a measuring unit (for example a stereotactic camera) to acquire the poses of the surface fiducial markers with respect to a coordinate system (WOR) of the measuring unit,
automatically computing a second coordinate transformation (^{IMA}T_{WOR}) from the coordinate system (WOR) of the measuring unit to the image coordinate system (IMA) via the coordinate system (SUR) of the surface fiducial markers,
measuring the pose of at least one adapter attached to an internal structure of the physical object, and automatically computing a third coordinate transformation (^{INN1}T_{SUR})from the coordinate system (SUR) of the surface fiducial markers to a coordinate system (INN₁) of the internal structure, and automatically combining the second coordinate transformation (^{IMA}T_{WOR}) with the first coordinate transformation (^{SUR}T_{IMA}) and with the third coordinate transformation (^{INN1}T_{SUR}) to create a coordinate transformation (^{INN1}T_{WOR}) from the coordinate system (WOR) of the measuring unit to the coordinate system (INN₁) of the internal structure, thereby allowing to measure the pose of the at least one adapter in the coordinate system (WOR) of the measuring unit.

According to a seventh aspect of the present invention, a computer program is disclosed, wherein the computer program comprises instructions to cause the medical system according to the present invention to execute the method according to the sixth aspect of the present invention.

Yet another aspect of the present invention relates to a computer-readable data carrier having stored thereon the computer program according to the seventh aspect of the present invention.

According to a further embodiment of the method according to the present invention (second aspect) additionally at least one adapter is attached to the internal structure by a pose-trackable surgical robotic device and wherein the at least one adapter can be tracked relative to the surface fiducial markers by the processing unit computing the coordinate transformations (^{SUR}T_{IMA} and ^{INN1}T_{SUR} and ^{INN1}T_{IMA}).

A further aspect of the present invention relates to a medical system, comprising:
a plurality of surface fiducial markers that are configured to be attached to an outer surface of a physical object in arbitrary spatial configurations with respect to each other,
at least one adapter that is configured to be attached to an internal structure of the physical object,
a medical imaging unit configured to generate a 3D image of said physical object and the surface fiducial markers attached to said outer surface within a predefined image coordinate system (IMA),
a processing unit configured for measuring each surface fiducial marker's pose within the 3D image and relative to the image coordinate system (IMA) and to compute a coordinate system (SUR) of the surface fiducial markers from the positions of the surface fiducial markers as well as a first coordinate transformation (^{SUR}T_{IMA}) between the image coordinate system (IMA) and the coordinate system (SUR) of the surface fiducial markers;
a measuring unit configured to acquire the poses of the surface fiducial markers with respect to a coordinate system (WOR) of the measuring unit when the respective surface fiducial marker is attached to said outer surface,
wherein the processing unit is further configured to compute a second coordinate system transformation (^{IMA}T_{WOR}) between the coordinate system (WOR) of the measuring unit and the image coordinate system (IMA) via the coordinate system (SUR) of the surface fiducial markers, thereby allowing to reference points on said outer surface to points within the 3D image, according to ^{IMA}T_{WOR}=(^{SUR}T_{IMA})^{-1,SUR}T_{WOR},
wherein the medical system, through its pose-trackable placement of the at least one adapter is configured to measure the pose of the at least one adapter relative to the surface fiducial markers when the at least one adapter is attached to the internal structure of said physical object, and wherein the processing unit is configured to compute a third coordinate transformation (^{INN1}T_{SUR}) between the coordinate system (SUR) of the surface fiducial markers and a coordinate system (INN₁) of the internal structure, and wherein the processing unit is configured to combine the second coordinate transformation (^{IMA}T_{WOR}) with the first coordinate transformation (^{SUR}T_{IMA}) and with the third coordinate transformation (^{INN1}T_{SUR}) to create a coordinate transformation (^{INN1}T_{WOR}) between the coordinate system (WOR) of the measuring unit and the coordinate system (INN₁) of the internal structure, according to ^{INN1}T_{SUR}*^{SUR}T_{IMA}*^{IMA}T_{WOR}=^{INN1}T_{WOR}, thereby allowing the medical system to measure the pose of the at least one adapter in the coordinate system (WOR) of the measuring unit.

According to an embodiment of the medical system, the medical system is configured to establish at least one further coordinate transformation (^{INN2}T_{WOR}) between the coordinate system (WOR) of the measuring unit and a coordinate system (INN₂) of a further internal structure of the physical object, wherein the medical system is configured to at least one of:
measure the pose of a further adapter relative to the surface fiducial markers when the further adapter is attached to the further internal structure of said physical object, and wherein the processing unit is configured to compute a further third coordinate transformation (^{INN2}T_{SUR}) between the coordinate system (SUR) of the surface fiducial markers and the coordinate system (INN₂) of the further internal structure, and wherein the processing unit is configured to combine the second coordinate transformation (^{IMA}T_{WOR}) with the first coordinate transformation (^{SUR}T_{IMA}) and with the further third coordinate transformation (^{INN2}T_{SUR}) to create the further coordinate transformation (^{INN2}T_{WOR}) between the coordinate system (WOR) of the measuring unit and the coordinate system (INN₂) of the further internal structure, thereby allowing to measure the pose of the further adapter in the coordinate system (WOR) of the measuring unit, or by
by combining the coordinate transformation (^{INN1}T_{WOR}) between the coordinate (WOR) of the measuring unit and the coordinate system (INN₁) of the internal structure with a coordinate transformation (^{INN2}T_{INN1}) between the coordinate system (INN₁) of the internal structure and a coordinate system (INN₂) of the further internal structure.

According to a further embodiment of the medical system, the medical system is configured to track said outer surface (2) using one of:
a. the surface fiducial markers, wherein particularly the medical system is configured to track the position or the pose of each individual surface fiducial marker, particularly using one of the following: an optical measurement principle, a video-optical measurement principle, an electromagnetic measurement principle, a time-of-flight measurement principle;
b. tracking of the outer surface by using one of: laser scanning of the outer surface, scanning the outer surface with structured light.

According to a further embodiment of the medical system, the medical system comprises different measurement modalities within one coordinate system to allow tracking of surface fiducial markers and the at least one adapter simultaneously using the different measurement modalities.

According to a further embodiment of the medical system, the medical system comprises a pose-trackable surgical robotic device configured to generate an access to the internal structure of the physical object and to deliver the at least one adapter to the internal structure and position the at least one adapter on the internal structure.

According to a further embodiment of the medical system, the medical system is configured to track several internal structures within the physical object independently and to establish and track several coordinate transformations between those internal structures and the outer surface, wherein at least one adapter has been delivered to each of the several internal structures by the pose-trackable surgical robotic device.

According to a further embodiment of the medical system, the medical system is configured to track several internal structures relative to one another and absolutely against the outer surface simultaneously.

According to a further embodiment of the medical system, the at least one adapter comprises a connecting portion that is configured to be releasably connected to a structure fiducial marker, and an anchoring portion that is configured to be attached to the internal structure so that an initially registered pose of the structure fiducial marker relative to the internal structure is reproduced upon re-connection of the structure fiducial marker to the adapter.

According to a further embodiment of the medical system, the anchoring portion comprises a thread on an outside of the anchoring portion for anchoring the at least one adapter to the internal structure by screwing the anchoring portion into a bore hole of the internal structure.

According to a further embodiment of the medical system, the connecting portion is configured to protrude from an outside of the internal structure when the anchoring portion is anchored to the internal structure, wherein the connecting portion comprises a plurality of image localization features that are integrated into the connecting portion, wherein particularly the respective image localization feature is a radiopaque marker.

According to a further embodiment of the medical system, the medical system is configured to intraoperatively acquire at least one image of the internal structure and the image localization features of the adapter and to locate the internal structure and the image localization features in the at least one intraoperatively acquired image and to compute a coordinate transformation (^{INN′}T_{WOR}) between the coordinate system (WOR) of the measuring unit and a coordinate system (INN′) of the internal structure.

According to a further embodiment of the medical system, the respective image localization feature is formed by a cylindrical rod, wherein particularly the rods are arranged obliquely with respect to one another.

According to a further embodiment of the medical system, the medical system is configured to compute the coordinate system (SUR) of the surface fiducial markers by using a position of a first surface fiducial marker as a center of the coordinate system (SUR) of the surface fiducial markers, wherein the medical system is further configured to use as a first coordinate axis (x) of the coordinate system (SUR) of the surface fiducial markers a normalized vector extending from the first surface fiducial marker to a second surface fiducial marker and as a second coordinate axis (y) a normalized vector extending from the first surface fiducial marker to a third surface fiducial marker and as a third coordinate axis (z) the cross product between the first and the second coordinate axis (x, y).

Yet another aspect of the present invention relates to a method for determining a coordinate transformation between a coordinate system (INN₁) of an internal structure inside a physical object and an image coordinate system (IMA) of a 3D image of the internal structure, wherein the method comprises the steps of:
(a) computing a first coordinate transformation (^{SUR}T_{IMA}) between a coordinate system (SUR) of surface fiducial markers attached to an outer side of the physical object and the image coordinate system (IMA) of the 3D image;
(b) computing a second coordinate transformation (^{INN1}T_{SUR}) between the coordinate system (SUR) of the surface fiducial markers attached to the physical object's outer side and a coordinate system (INN₁) of an internal structure inside the physical object;
(c) automatically extending the coordinate transformation (^{SUR}T_{IMA}) computed in step (a) by the coordinate transformation (^{INN1}T_{SUR}) computed in step (b) to create a coordinate transformation (^{INN1}T_{IMA}) between the image coordinate system (IMA) of the 3D image and the coordinate system (INN₁) of the internal structure.

According to a further embodiment of the method, the outer surface is tracked by one of:
a. individually tracking the pose or position of the surface fiducial markers, particularly by means of one of optical tracking, video-optical tracking, electromagnetic tracking, time-of-flight tracking;
b. tracking of the outer surface by means of laser scanning or scanning with structured light.

Furthermore, according to a further embodiment of the method, additionally at least one adapter is attached to the internal structure by a pose-trackable surgical robotic device and wherein the at least one adapter can be tracked relative to the surface fiducial markers by the processing unit computing the coordinate transformations (^{SUR}T_{IMA} and ^{INN1}T_{SUR} and ^{INN1}T_{IMA}).

In the following embodiments as well as further features and advantages of the present invention are described with reference to the Figures, wherein

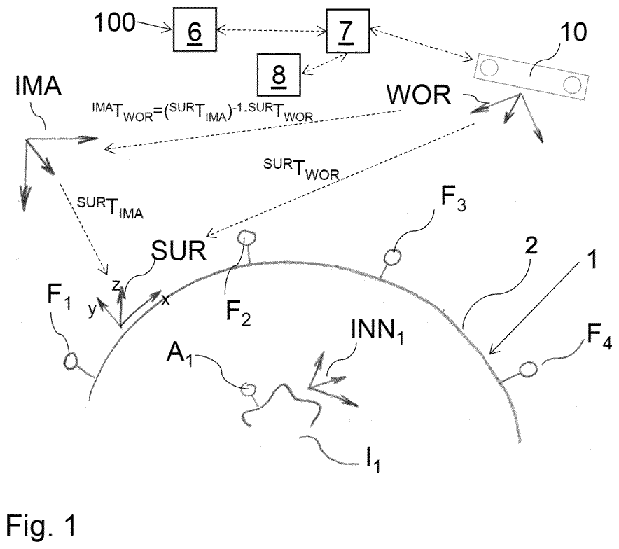

FIG. 1 shows a schematic illustration of a medical system according to the present invention;

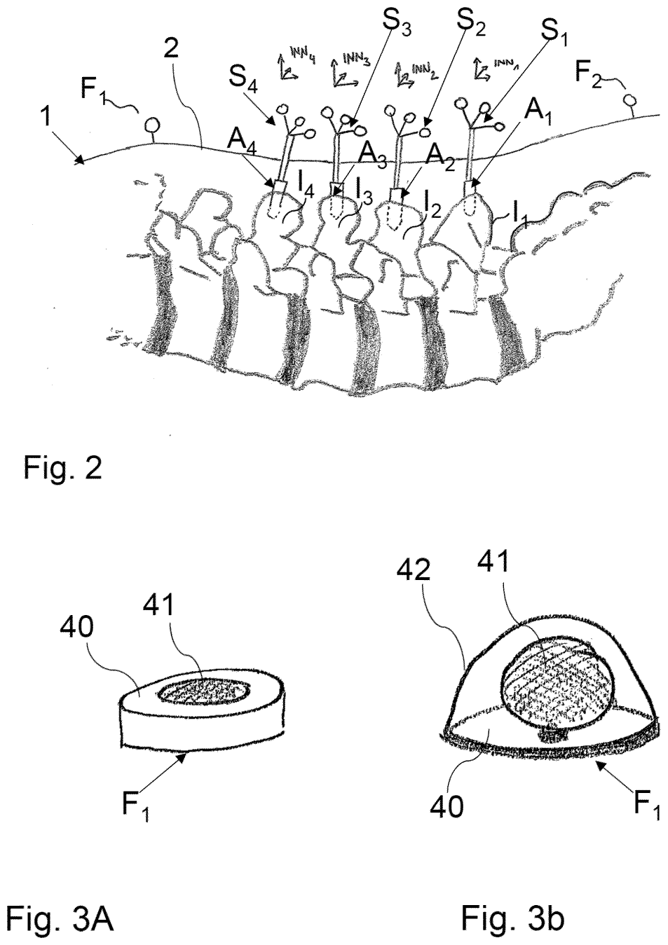

FIG. 2 shows a detailed illustration of the adapters for holding the structure fiducial markers of the system according to FIG. 1 when attached to the internal structure (e.g. vertebrae) of the patient,

FIG. 3A shows an illustration of an embodiment of a surface fiducial marker;

FIG. 3B shows an illustration of a further embodiment of a surface fiducial marker;

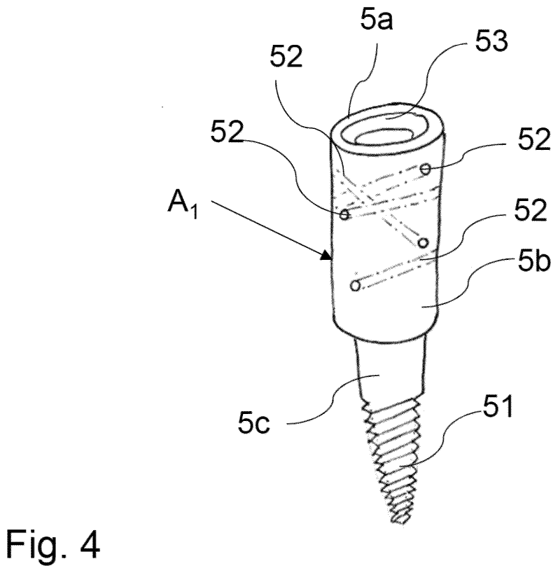

FIG. 4 shows an embodiment of an adapter of the medical system that is configured to be attached to an internal structure (e.g. vertebra) and allows for a releasable but reproducible connection of a structure fiducial marker to the adapter;

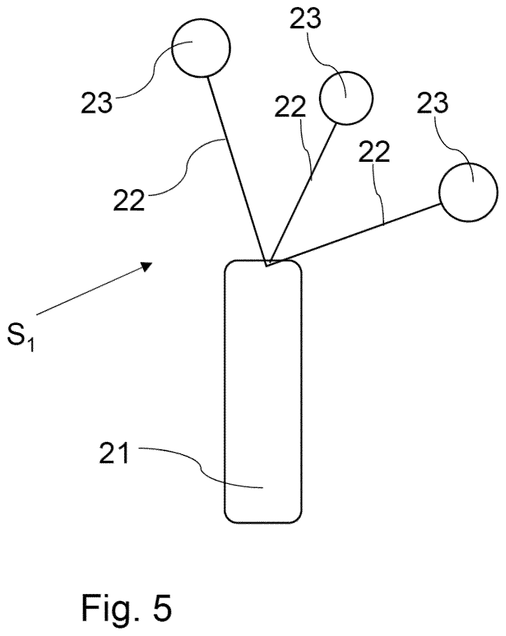

FIG. 5 shows an embodiment of a structure fiducial marker that can be connected to the adapter shown in FIG. 4; and

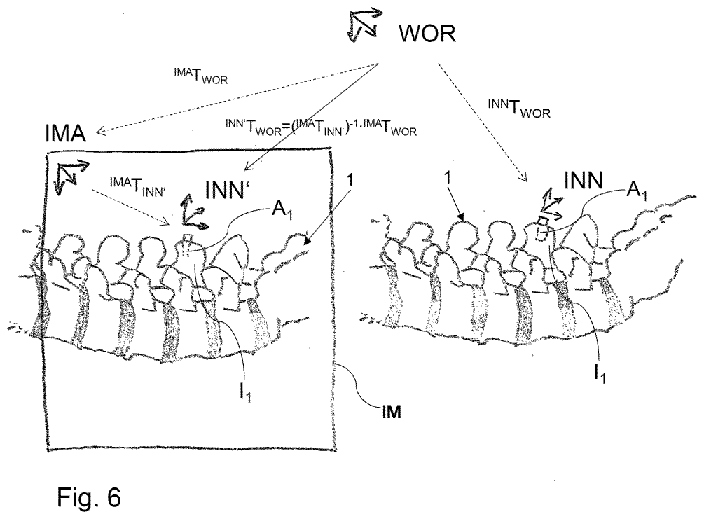

FIG. 6 shows refining of a coordinate transformation between a coordinate system of an internal structure and the coordinate system of the measuring unit using an intraoperatively obtained image.

FIG. 1 shows an embodiment of a medical system 100 according to the present invention that allows for determination of a geometric transformation between an internal structure 11 such as a spinal vertebra inside a physical object 1, i.e., the body 1 of the patient, and a 3D image of the object/body 1. To this end, the medical system 100 comprises a measuring unit 10, a processing unit 7, a medical imaging unit 6 (e.g. a CT scanner or an MRI device) and preferably also a surgical robotic device 8. It is well understood that the optical tracking system may be used to track the pose of surgical instruments, the surgical robotic device 8 and adapter devices. The processing unit 7 can comprise any suitable computer. The processing unit 7 can be a stand-alone unit, but may also be an integral part of another component of the system 100. The processing unit 7 preferably comprises interfaces to connect to the medical imaging unit 6 (e.g. to receive a 3D image of the patient) and particularly to the surgical robotic device 8, e.g. for controlling the latter. Furthermore, the processing unit 7 can comprise an interface for connecting to the measuring unit 10.

Furthermore, the medical system 100 comprises a plurality of surface fiducial markers Fᵢ (wherein i is a natural number that labels the surface fiducial markers) that can each be configured as shown in FIG. 3A below. The surface fiducial markers Fᵢ are each designed to be attached to an outer surface 2 of a physical object 1 (e.g. skin 2 of a body 1 of a patient) in arbitrary spatial configurations with respect to each other.

Further, the medical system 100 comprises at least one adapter A₁ for providing reproducible connection of an associated structure fiducial marker S₁ to the adapter A₁ (several such adapters Aᵢ/structure fiducial marker Sᵢ are used in case several internal structures Iᵢ shall be tracked, wherein i is again a natural number that labels the adapters, surface fiducial markers, and internal structures, respectively), wherein the adapter A₁ is configured to be attached to an internal structure I₁ of the physical object 1. An embodiment of a preferred adapter A₁ will be described in conjunction with FIG. 4 in detail further below.

Particularly, the medical imaging unit 6 is configured to generate a 3D image of said physical object 1 and the surface fiducial markers Fᵢ attached to said outer surface 2 of the object 1 with respect to an image coordinate system IMA.

With help of the processing unit 7 (for example a computer on which a suitable software is executed) each surface fiducial marker's pose is measured within the 3D image and relative to the image coordinate system IMA. This may be carried out automatically or guided by a user/physician. Further, the processing unit 7 is configured to compute a coordinate system SUR of the surface fiducial markers Fᵢ from the positions of the surface fiducial markers as well as a first coordinate transformation ^{SUR}T_{IMA} between the image coordinate system IMA and the coordinate system SUR of the surface fiducial markers Fᵢ.

Further, the measuring unit 10 (for example a stereotactic camera) is configured to acquire the poses of the surface fiducial markers Fᵢ with respect to a coordinate system WOR of the measuring unit 10 when the respective surface fiducial marker Fᵢ is attached to said outer surface 2 of the object 1 as shown in FIG. 1.

Regarding this measurement unit 10, the processing unit 7 is further configured to compute a second coordinate system transformation ^{IMA}T_{WOR} between the coordinate system WOR of the measuring unit 10 and the image coordinate system IMA via the coordinate system SUR of the surface fiducial markers Fᵢ, thereby allowing for reference between points on said outer surface 2 of the object 1 to points within the 3D image or model generated with help of the medical imaging unit 6.

The system 1 is now configured to measure the pose of the at least one adapter A₁ attached to the internal structure Ii relative to the surface fiducial markers Fᵢ. Particularly a surgical robotic device 8 can measure the pose of the adapter A₁ upon attaching the adapter A₁ to the internal structure I₁ by means of the robotic device 8 of the system 100. The skilled person will understand that the pose of the robotic device 8 may also be measured through a tracking camera in conjunction with tracking markers positioned on the robotic device 8 (e.g., on the joints of the robotic device 8) or may be deduced through the medical imaging unit 6 providing data on the position of surgical instruments attached to the robotic device 8. In this regard, the coordinate system of the tracking camera may measure both the position of an end effector of the robotic device 8 in space as well as the position of the surface fiducial markers on the patient, thus allowing the deduction of the position of the end effector with respect to the patient coordinate system. Further, the processing unit 7 is configured to compute a third coordinate transformation ^{INN1}T_{SUR} between the coordinate system SUR of the surface fiducial markers Fᵢ and a coordinate system INN₁ of the internal structure I₁.

The processing unit 7 then combines the second coordinate transformation ^{IMA}T_{WOR} with the first coordinate transformation ^{SUR}T_{IMA} and with the third coordinate transformation ^{INNA1}T_{SUR}, for example by multiplying the associated matrices:

^{INN1}T_{SUR}^{SUR}T_{IMA}^{IMA}T_{WOR}=^{INN1}T_{WOR}.

to create a final coordinate transformation represented by the matrix ^{INN1}T_{WOR}, thereby allowing to measure the pose of the adapter A₁ (or of a structure fiducial marker S₁ connected to the adapter A₁) in the coordinate system WOR.

In order to be able to attach the structure fiducial marker S₁ in an efficient and reproducible manner to the respective internal structure Iᵢ, the medical system 100 preferably comprises the at least one adapter A₁ as shown in FIG. 4. The system 100 can comprise several adapters Aᵢ and associated structure fiducial markers Sᵢ).

The at least one adapter A₁ comprises a connecting portion 5b that is configured to be releasably connected to the at least one structure fiducial marker S₁ (cf. e.g. FIG. 5). For this, the adapter A₁ may comprise a connector 53 e.g. formed by an opening 53 arranged in a face side 5a of the connecting portion 5b of the adapter 5. The fiducial marker Si can be configured to engage with the connector/opening 53 to generate a releasable mechanical connection between the marker S₁ and the adapter A₁ (the structure fiducial marker S₁ is not indicated in FIG. 2 but shown in FIG. 5). Furthermore, the adapter A₁ comprises an anchoring portion 5c that is configured to be attached to the internal structure I₁. In case the system 100 comprises several adapters A₁ and structure fiducial markers Sᵢ, these adapters and structure fiducial markers can be designed as the adapter A₁ and marker S1, respectively.

Due to the specific design of the at least one adapter A₁, an initially registered pose of the structure fiducial marker S₁ (when connected to the adapter A₁) relative to the internal structure I₁ is reproduced upon re-connection of the released structure fiducial marker 20 to the adapter A₁.

For anchoring the anchoring portion 5c in the internal structure (e.g. bone, particularly vertebra) I₁ the adapter A₁ comprises a thread 51 formed on an outside of the anchoring portion 5c. Thus, the anchoring portion 5c can be screwed into a bore hole provide in the internal structure I₁ (e.g. by way of the pose-trackable surgical robotic device 8). Particularly, the anchoring portion 5c is tapered to form a pointed end of the anchoring portion 5c which improves insertion into the bore hole.

Particularly, the connecting portion 5b is configured to extend along an outside of the internal structure I₁ when the anchoring portion 5c is anchored to the internal structure I₁ as described above, wherein the connecting portion 5c comprises a plurality of image localization features 52 that are integrated into the connecting portion 5b, wherein particularly the respective image localization feature 52 is a radiopaque marker. In an embodiment, the respective image localization features 52 is formed by a cylindrical rod, wherein the rods are arranged obliquely with respect to one another as indicated in FIG. 4.

An embodiment of a structure fiducial marker S₁ that can e.g. be used for tracking during surgery and is not necessary for the registering process according to the present invention is shown in FIG. 5. According thereto, the marker S₁ comprises a base portion 21 that is configured to engage with the connector 53 of the adapter A₁ so as to releasably connect the marker S₁ to the adapter A₁. Furthermore, the marker S₁ can comprise several (e.g. three) individivally trackable fiducial elements 23, particularly in the form of spheres, that can be connected to the base 21 portion via arms 22.

Furthermore, FIG. 3A shows an embodiment of a surface fiducial marker F₁ that comprises a flat cylindrical body 40 into which a visually trackable fiducial element 41 cab be embedded.

FIG. 3B shows an alternative embodiment of a surface fiducial marker F₁, wherein here the fiducial element 41 is formed by a retroreflective sphere, wherein the tracking element 41 can be connected to a circular base 40 and can further be covered by a transparent cover 42 connected to the base 40.

Furthermore, FIG. 6 shows that the transformation ^{INN1}T_{WOR} can be further refined or replaced via an intraoperative imaging method, wherein the previously existing coordinate system transformation ^{INN1}T_{WOR} is refined by algorithmically locating the internal structure I₁ and the image localization features 52 of the adapter A₁ in the resulting imagery and by computing a subsequent incremental registration transformation ^{INN′}T_{WOR}. Particularly, the medical system 100 can be configured to compute a coordinate transformation ^{IMA}T_{INN′} from a coordinate system INN′ of the internal structure Ii in an intraoperatively obtained image IM to an image coordinate system IMA of the intraoperatively obtained image IM as indicated in FIG. 6, and by combining this transformation ^{IMA}T_{INN′} with the already computed coordinate transformation ^{IMA}T_{WOR}

^{INN′}T_{WOR}=(^{IMA}T_{INN′})^{-1,IMA}T_{WOR}.

to determine the refined coordinate transformation ^{INN′}T_{WOR} from the coordinate system WOR of the measuring unit 10 to the coordinate system INN′ of the internal structure.

The medical system 100 according to the present invention as described herein is particularly suited to perform the methods according to the present invention.

The non-surgical methods allow for determination of a coordinate transformation between a coordinate system INN₁ of an internal structure I₁ as shown in FIG. 1 inside a physical object 1 and a coordinate system of a 3D image thereof (e.g. obtained with the medical imaging unit 6), wherein generating this transformation is achieved stepwise by:
(a) computing an initial coordinate transformation ^{SUR}T_{IMA} between a coordinate system SUR of surface fiducial markers Fᵢ (i=1, 2, . . . ) attached to the outer side 2 of the physical object 1 and the 3D image (i.e. registration);
(b) computing a coordinate transformation ^{INN1}T_{SUR} between the coordinate system SUR of the surface fiducial markers Fᵢ (i=1, 2, . . . ) attached to the physical object's outer side 2 and a coordinate system INN₁ of an internal structure I₁ to which at least one adapter A₁ is already attached inside the physical object 1. Particularly, the adapter A₁ serves for connecting a structure fiducial marker S₁ (cf. FIG. 5) to the adapter A₁ in a reproducible fashion that can later be tracked (e.g. during surgery); and
(c) extending the coordinate transformation computed in step (a) by the coordinate transformation computed in step (b) to create a coordinate transformation between the image coordinate system IMA of the 3D image and the coordinate system INN₁ associated with the internal structure I₁.

Particularly, in an embodiment, this method may be employed with respect to internal structures of a patient formed by spinal vertebrae Iᵢ (i=1, 2, . . . ), wherein the physical object 1 is an upper body of the patient. However, the present invention can also be applied to any other internal structure that allows placement of the adapters or structural fiducial markers.

Particularly, using the method according to the present invention, the following procedure relating to the tracking of vertebrae (or other internal structures) Iᵢ can be carried out:
1) Attaching surface fiducial markers Fᵢ visible in an 3D image created by a medical imaging unit 6 (e.g. CT or MRI) and in a measurement unit (e.g. such as a stereotactic camera) 10 to the outer surface 2 of a body of a subject (i.e. skin)
2) Producing a 3D image using the medical imaging unit 6 and determination of the surface fiducial marker's position or pose within the image coordinate system IMA. Computation of a (for example perpendicular right-hand) coordinate system SUR from the surface fiducial marker's positions.
3) Setup of tracking and subsequent tracking of the outer surface 2 via said surface fiducial markers Fᵢ (particularly based on the assumption that no deformation is present as long no surgical intervention (cut, screw placement etc.) is carried out on the body/outer surface of the subject.
4) Transfer of the 3D image to a software system (e.g. processing unit) 7 and marking of a suitable place to drill and insert an adapter A₁ for a structure fiducial marker S₁ into the internal structure (e.g. vertebra of subject) I₁. This step may be repeated for as often as necessary to prepare the method according to the present invention beforehand, but does not constitute a step of the claimed method.
5) Use of a suitable surgical device or control of a pose-trackable surgical robotic device 8 to drill a hole into vertebra I₁ at the planned position and orientation (according to step 4). Particularly, minimal to no distortion of the surface fiducial markers Fᵢ is present (also this step does not constitute a step of the claimed method).
6) Attachment of the adapter A₁ to the internal structure/vertebra I₁ (also this step does not constitute a step of the claimed method). Particularly, the pose of the adapter A₁ is known to the system 100 via the pose-trackable surgical robotic device 8 that can measure the exact pose of the adapter A₁ (and thus of a marker 20 connected to the adapter later on) upon attaching the adapter A₁.
7) A tracking system (e.g. comprising measurement unit 10) records relative positions or poses of surface fiducial markers Fᵢ and of the adapter A₁ or of a structure fiducial marker S₁ connected to the adapter A₁, and medical system 100 computes relative geometric transformation between the surface and adapter A₁/structure fiducial marker S₁. Registration between tracked internal structure (e.g. vertebra) I₁ and 3D image data set is then made available.
8) Repeat steps 4-7 for each internal structure (e.g. vertebra) Iᵢ (i>1) and corresponding adapters Aᵢ (i>1).

### REFERENCE NUMERALS

1 physical object (e.g. body or body portion of patient)
2 outer surface of physical object
Iᵢ i-th internal structure of the object, not otherwise accessible from the outside
Fᵢ i-th surface fiducial marker attached to the outer surface
Aᵢ i-th adapter attached to the objects internal structure (after creation of access) connectable to structure fiducial marker
5a face side
5b connecting portion
5c anchoring portion
6 medical imaging device (e.g. CT scanner or MRI device)
7 processing unit
8 surgical robot
10 measurement unit
IMA image coordinate system (of the 3D image/model)
WOR coordinate system of the measurement unit
SUR coordinate system of the surface fiducial markers
INN₁ coordinate system of the objects internal structure
INN′ coordinate system of internal structure
Sᵢ i-th structure fiducial marker for internal structures
51 thread
52 image localization feature
53 opening/connector for connecting structure fiducial marker to adapter
100 medical system

1. A medical system (100), comprising:
a plurality of surface fiducial markers (Fᵢ) that are configured to be attached to an outer surface (2) of a physical object (1) in arbitrary spatial configurations with respect to each other,
at least one adapter (A₁) that is configured to be attached to an internal structure (I₁) of the physical object (1),
a medical imaging unit (6) configured to generate a 3D image of said physical object (1) and the surface fiducial markers (Fᵢ) attached to said outer surface (2) within a predefined image coordinate system (IMA),
a processing unit (7) configured for measuring each surface fiducial marker's pose within the 3D image and relative to the image coordinate system (IMA) and to compute a coordinate system (SUR) of the surface fiducial markers (Fᵢ) from the positions of the surface fiducial markers (Fᵢ) as well as a first coordinate transformation (^{SUR}T_{IMA}) between the image coordinate system (IMA) and the coordinate system (SUR) of the surface fiducial markers (Fᵢ);
a measuring unit (10) configured to acquire the poses of the surface fiducial markers (Fᵢ) with respect to a coordinate system (WOR) of the measuring unit (10) when the respective surface fiducial marker (Fᵢ) is attached to said outer surface (2),
wherein the processing unit (7) is further configured to compute a second coordinate system transformation (^{IMA}T_{WOR}) between the coordinate system (WOR) of the measuring unit (10) and the image coordinate system (IMA) via the coordinate system (SUR) of the surface fiducial markers (Fᵢ), thereby allowing to reference points on said outer surface (2) to points within the 3D image,
wherein the medical system (100), through its pose-trackable placement of the at least one adapter (A₁) is configured to measure the pose of the at least one adapter (A₁) relative to the surface fiducial markers (Fᵢ) when the at least one adapter (A₁) is attached to the internal structure (I₁) of said physical object (1), and wherein the processing unit (7) is configured to compute a third coordinate transformation (^{INN1}T_{SUR}) between the coordinate system (SUR) of the surface fiducial markers (Fᵢ) and a coordinate system (INN₁) of the internal structure (I₁), and wherein the processing unit (7) is configured to combine the second coordinate transformation (^{IMA}T_{WOR}) with the first coordinate transformation (^{SUR}T_{IMA}) and with the third coordinate transformation (^{INN1}T_{SUR}) to create a coordinate transformation (^{INN1}T_{WOR}) between the coordinate system (WOR) of the measuring unit (10) and the coordinate system (INN₁) of the internal structure (I₁), thereby allowing the medical system (100) to measure the pose of the at least one adapter (A₁) in the coordinate system (WOR) of the measuring unit (10).

2. The system according to claim 1, wherein the medical system (100) is configured to establish at least one further coordinate transformation (^{INN2}T_{WOR}) between the coordinate system (WOR) of the measuring unit (10) and a coordinate system (INN₂) of a further internal structure (I₂) of the physical object (1), wherein the medical system (100) is configured to at least one of:
measure the pose of a further adapter (A₂) relative to the surface fiducial markers (Fᵢ) when the further adapter (A₂) is attached to the further internal structure (I₂) of said physical object (1), and wherein the processing unit (7) is configured to compute a further third coordinate transformation (^{INN2}T_{SUR}) between the coordinate system (SUR) of the surface fiducial markers (Fᵢ) and the coordinate system (INN₂) of the further internal structure (I₂), and wherein the processing unit (7) is configured to combine the second coordinate transformation (^{IMA}T_{WOR}) with the first coordinate transformation (^{SUR}T_{IMA}) and with the further third coordinate transformation (^{INN2}T_{SUR}) to create the further coordinate transformation (^{INN2}T_{WOR}) between the coordinate system (WOR) of the measuring unit (10) and the coordinate system (INN₂) of the further internal structure (I₂), thereby allowing to measure the pose of the further adapter (A₂) in the coordinate system (WOR) of the measuring unit (10), or by
by combining the coordinate transformation (^{INN1}T_{WOR}) between the coordinate (WOR) of the measuring unit (10) and the coordinate system (INN₁) of the internal structure (I₁) with a coordinate transformation (^{INN2}T_{INN1}) between the coordinate system (INN₁) of the internal structure (I₁) and a coordinate system (INN₂) of the further internal structure (12).

3. The medical system according to claim 1 or 2, wherein the medical system (100) is configured to track said outer surface (2) using one of:
c. the surface fiducial markers (Fᵢ), wherein particularly the medical system (100) is configured to track the position or the pose of each individual surface fiducial marker (Fᵢ), particularly using one of the following: an optical measurement principle, a video-optical measurement principle, an electromagnetic measurement principle, a time-of-flight measurement principle;
d. tracking of the outer surface (2) by using one of: laser scanning of the outer surface (2), scanning the outer surface (2) with structured light.

4. The medical system according to one of the preceding claims, wherein the medical system (100) comprises different measurement modalities within one coordinate system to allow tracking of surface fiducial markers (Fᵢ) and the at least one adapter (A₁) simultaneously using the different measurement modalities.

5. The medical system according to one of the preceding claims, wherein the medical system (100) comprises a pose-trackable surgical robotic device (8) configured to generate an access to the internal structure (I₁) of the physical object (1) and to deliver the at least one adapter (A₁) to the internal structure (3) and position the at least one adapter (A₁) on the internal structure (3).

6. The medical system according to one of the preceding claims, wherein the medical system (100) is configured to track several internal structures (I₁) within the physical object (1) independently and to establish and track several coordinate transformations between those internal structures (Iᵢ) and the outer surface (2), wherein at least one adapter (A₁) has been delivered to each of the several internal structures (Iᵢ) by the pose-trackable surgical robotic device (8).

7. The medical system according to one of the preceding claims, wherein the medical system (100) is configured to track several internal structures (Iᵢ) relative to one another and absolutely against the outer surface (2) simultaneously.

8. The medical system according to one of the preceding claims, wherein the at least one adapter (A₁) comprises a connecting portion (5b) that is configured to be releasably connected to a structure fiducial marker (S₁), and an anchoring portion (5c) that is configured to be attached to the internal structure (I₁) so that an initially registered pose of the structure fiducial marker (S₁) relative to the internal structure (I₁) is reproduced upon re-connection of the structure fiducial marker (S₁) to the adapter (A₁).

9. The medical system according to claim 8, wherein the anchoring portion (5c) comprises a thread (51) on an outside of the anchoring portion (5c) for anchoring the at least one adapter (A₁) to the internal structure (I₁) by screwing the anchoring portion (5c) into a bore hole of the internal structure (I₁).

10. The medical system according to claim 8 or 9, wherein the connecting portion (5b) is configured to protrude from an outside of the internal structure (I₁) when the anchoring portion (5c) is anchored to the internal structure (I₁), wherein the connecting portion (5b) comprises a plurality of image localization features (52) that are integrated into the connecting portion (5b), wherein particularly the respective image localization feature (52) is a radiopaque marker.

11. The medical system according to claim 10, wherein the medical system (100) is configured to intraoperatively acquire at least one image (IM) of the internal structure (I₁) and the image localization features (52) of the adapter (A₁) and to locate the internal structure (I₁) and the image localization features (52) in the at least one intraoperatively acquired image (IM) and to compute a coordinate transformation (^{INN′}T_{WOR}) between the coordinate system (WOR) of the measuring unit (10) and a coordinate system (INN′) of the internal structure (I₁).

12. The medical system according to claim 10 or 11, wherein the respective image localization feature (52) is formed by a cylindrical rod, wherein particularly the rods (52) are arranged obliquely with respect to one another.

13. The medical system according to one of the preceding claims, wherein the medical system (100) is configured to compute the coordinate system (SUR) of the surface fiducial markers (Fᵢ) by using a position of a first surface fiducial marker (F₁) as a center of the coordinate system (SUR) of the surface fiducial markers (Fᵢ), wherein the medical system is further configured to use as a first coordinate axis (x) of the coordinate system (SUR) of the surface fiducial markers (Fᵢ) a normalized vector extending from the first surface fiducial marker (F₁) to a second surface fiducial marker (F₂) and as a second coordinate axis (y) a normalized vector extending from the first surface fiducial marker (F₁) to a third surface fiducial marker (F₃) and as a third coordinate axis (z) the cross product between the first and the second coordinate axis (x, y).

14. A method for determining a coordinate transformation between a coordinate system (INN₁) of an internal structure (I₁) inside a physical object (1) and an image coordinate system (IMA) of a 3D image of the internal structure (I₁), wherein the method comprises the steps of:
(a) computing a first coordinate transformation (^{SUR}T_{IMA}) between a coordinate system (SUR) of surface fiducial markers (Fᵢ) attached to an outer side (2) of the physical object (1) and the image coordinate system (IMA) of the 3D image;
(b) computing a second coordinate transformation (^{INN1}T_{SUR}) between the coordinate system (SUR) of the surface fiducial markers (Fᵢ) attached to the physical object's outer side (2) and a coordinate system (INN₁) of an internal structure (I₁) inside the physical object (1);
(c) extending the coordinate transformation (^{SUR}T_{IMA}) computed in step (a) by the coordinate transformation (^{INN1}T_{SUR}) computed in step (b) to create a coordinate transformation (^{INN1}T_{IMA}) between the image coordinate system (IMA) of the 3D image and the coordinate system (INN₁) of the internal structure (I₁).

15. The method according to claim 14, wherein the outer surface (2) is tracked by one of:
a. individually tracking the pose or position of the surface fiducial markers (Fᵢ), particularly by means of one of optical tracking, video-optical tracking, electromagnetic tracking, time-of-flight tracking;
b. tracking of the outer surface (2) by means of laser scanning or scanning with structured light.

16. The method according to claim 14, wherein additionally at least one adapter (A₁) is attached to the internal structure (Iᵢ) by a pose-trackable surgical robotic device (8) and wherein the at least one adapter (A₁) can be tracked relative to the surface fiducial markers (Fᵢ) by the processing unit (7) computing the coordinate transformations (^{SUR}T_{IMA} and ^{INN1}T_{SUR} and ^{INN1}T_{IMA}).

17. A medical system (100), comprising:
a plurality of surface fiducial markers (Fᵢ) that are configured to be attached to an outer surface (2) of a physical object (1) in arbitrary spatial configurations with respect to each other,
at least one adapter (A₁) that is configured to be attached to an internal structure (I₁) of the physical object (1),
a medical imaging unit (6) configured to generate a 3D image of said physical object (1) and the surface fiducial markers (Fᵢ) attached to said outer surface (2) within a predefined image coordinate system (IMA),
a processing unit (7) configured for measuring each surface fiducial marker's pose within the 3D image and relative to the image coordinate system (IMA) and to compute a coordinate system (SUR) of the surface fiducial markers (Fᵢ) from the positions of the surface fiducial markers (Fᵢ) as well as a first coordinate transformation (^{SUR}T_{IMA}) between the image coordinate system (IMA) and the coordinate system (SUR) of the surface fiducial markers (Fᵢ);
a measuring unit (10) configured to acquire the poses of the surface fiducial markers (Fᵢ) with respect to a coordinate system (WOR) of the measuring unit (10) when the respective surface fiducial marker (Fᵢ) is attached to said outer surface (2),
wherein the processing unit (7) is further configured to compute a second coordinate system transformation (^{IMA}T_{WOR}) between the coordinate system (WOR) of the measuring unit (10) and the image coordinate system (IMA) via the coordinate system (SUR) of the surface fiducial markers (Fᵢ), thereby allowing to reference points on said outer surface (2) to points within the 3D image, according to ^{IMA}T_{WOR}=(^{SUR}T_{IMA})^{-1,SUR}T_{WOR},
wherein the medical system (100), through its pose-trackable placement of the at least one adapter (A₁) is configured to measure the pose of the at least one adapter (A₁) relative to the surface fiducial markers (Fᵢ) when the at least one adapter (A₁) is attached to the internal structure (I₁) of said physical object (1), and wherein the processing unit (7) is configured to compute a third coordinate transformation (^{INN1}T_{SUR}) between the coordinate system (SUR) of the surface fiducial markers (Fᵢ) and a coordinate system (INN₁) of the internal structure (I₁), and wherein the processing unit (7) is configured to combine the second coordinate transformation (^{IMA}T_{WOR}) with the first coordinate transformation (^{SUR}T_{IMA}) and with the third coordinate transformation (^{INN1}T_{SUR}) to create a coordinate transformation (^{INN1}T_{WOR}) between the coordinate system (WOR) of the measuring unit (10) and the coordinate system (INN₁) of the internal structure (I₁), according to ^{INN1}T_{SUR}*^{SUR}T_{IMA}*^{IMA}T_{WOR}=^{INN1}T_{WOR}, thereby allowing the medical system (100) to measure the pose of the at least one adapter (A₁) in the coordinate system (WOR) of the measuring unit (10).

18. The system according to claim 17, wherein the medical system (100) is configured to establish at least one further coordinate transformation (^{INN2}T_{WOR}) between the coordinate system (WOR) of the measuring unit (10) and a coordinate system (INN₂) of a further internal structure (12) of the physical object (1), wherein the medical system (100) is configured to at least one of:
measure the pose of a further adapter (A₂) relative to the surface fiducial markers (Fᵢ) when the further adapter (A₂) is attached to the further internal structure (I₂) of said physical object (1), and wherein the processing unit (7) is configured to compute a further third coordinate transformation (^{INN2}T_{SUR}) between the coordinate system (SUR) of the surface fiducial markers (Fᵢ) and the coordinate system (INN₂) of the further internal structure (I₂), and wherein the processing unit (7) is configured to combine the second coordinate transformation (^{IMA}T_{WOR}) with the first coordinate transformation (^{SUR}T_{IMA}) and with the further third coordinate transformation (^{INN2}T_{SUR}) to create the further coordinate transformation (^{INN2}T_{WOR}) between the coordinate system (WOR) of the measuring unit (10) and the coordinate system (INN₂) of the further internal structure (I₂), thereby allowing to measure the pose of the further adapter (A₂) in the coordinate system (WOR) of the measuring unit (10), or by
by combining the coordinate transformation (^{INN1}T_{WOR}) between the coordinate (WOR) of the measuring unit (10) and the coordinate system (INN₁) of the internal structure (I₁) with a coordinate transformation (^{INN2}T_{INN1}) between the coordinate system (INN₁) of the internal structure (I₁) and a coordinate system (INN₂) of the further internal structure (I₂).

19. The medical system according to claim 17 or 18, wherein the medical system (100) is configured to track said outer surface (2) using one of:
e. the surface fiducial markers (Fᵢ), wherein particularly the medical system (100) is configured to track the position or the pose of each individual surface fiducial marker (Fᵢ), particularly using one of the following: an optical measurement principle, a video-optical measurement principle, an electromagnetic measurement principle, a time-of-flight measurement principle;
f. tracking of the outer surface (2) by using one of: laser scanning of the outer surface (2), scanning the outer surface (2) with structured light.

20. The medical system according to one of the preceding claims, wherein the medical system (100) comprises different measurement modalities within one coordinate system to allow tracking of surface fiducial markers (Fᵢ) and the at least one adapter (A₁) simultaneously using the different measurement modalities.

21. The medical system according to one of the preceding claims, wherein the medical system (100) comprises a pose-trackable surgical robotic device (8) configured to generate an access to the internal structure (I₁) of the physical object (1) and to deliver the at least one adapter (A₁) to the internal structure (3) and position the at least one adapter (A₁) on the internal structure (3).

22. The medical system according to one of the preceding claims, wherein the medical system (100) is configured to track several internal structures (10 within the physical object (1) independently and to establish and track several coordinate transformations between those internal structures (Iᵢ) and the outer surface (2), wherein at least one adapter (A₁) has been delivered to each of the several internal structures (Iᵢ) by the pose-trackable surgical robotic device (8).

23. The medical system according to one of the preceding claims, wherein the medical system (100) is configured to track several internal structures (Iᵢ) relative to one another and absolutely against the outer surface (2) simultaneously.

24. The medical system according to one of the preceding claims, wherein the at least one adapter (A₁) comprises a connecting portion (5b) that is configured to be releasably connected to a structure fiducial marker (S₁), and an anchoring portion (5c) that is configured to be attached to the internal structure (I₁) so that an initially registered pose of the structure fiducial marker (S₁) relative to the internal structure (I₁) is reproduced upon re-connection of the structure fiducial marker (S₁) to the adapter (A₁).

25. The medical system according to claim 24, wherein the anchoring portion (5c) comprises a thread (51) on an outside of the anchoring portion (5c) for anchoring the at least one adapter (A₁) to the internal structure (I₁) by screwing the anchoring portion (5c) into a bore hole of the internal structure (I₁).

26. The medical system according to claim 24 or 25, wherein the connecting portion (5b) is configured to protrude from an outside of the internal structure (I₁) when the anchoring portion (5c) is anchored to the internal structure (I₁), wherein the connecting portion (5b) comprises a plurality of image localization features (52) that are integrated into the connecting portion (5b), wherein particularly the respective image localization feature (52) is a radiopaque marker.

27. The medical system according to claim 26, wherein the medical system (100) is configured to intraoperatively acquire at least one image (IM) of the internal structure (I₁) and the image localization features (52) of the adapter (A₁) and to locate the internal structure (I₁) and the image localization features (52) in the at least one intraoperatively acquired image (IM) and to compute a coordinate transformation (^{INN′}T_{WOR}) between the coordinate system (WOR) of the measuring unit (10) and a coordinate system (INN′) of the internal structure (I₁).

28. The medical system according to claim 26 or 27, wherein the respective image localization feature (52) is formed by a cylindrical rod, wherein particularly the rods (52) are arranged obliquely with respect to one another.

29. The medical system according to one of the preceding claims, wherein the medical system (100) is configured to compute the coordinate system (SUR) of the surface fiducial markers (Fᵢ) by using a position of a first surface fiducial marker (F₁) as a center of the coordinate system (SUR) of the surface fiducial markers (Fᵢ), wherein the medical system is further configured to use as a first coordinate axis (x) of the coordinate system (SUR) of the surface fiducial markers (Fᵢ) a normalized vector extending from the first surface fiducial marker (F₁) to a second surface fiducial marker (F₂) and as a second coordinate axis (y) a normalized vector extending from the first surface fiducial marker (F₁) to a third surface fiducial marker (F₃) and as a third coordinate axis (z) the cross product between the first and the second coordinate axis (x, y).

30. A method for determining a coordinate transformation between a coordinate system (INN₁) of an internal structure (I₁) inside a physical object (1) and an image coordinate system (IMA) of a 3D image of the internal structure (I₁), wherein the method comprises the steps of:
(a) computing a first coordinate transformation (^{SUR}T_{IMA}) between a coordinate system (SUR) of surface fiducial markers (Fᵢ) attached to an outer side (2) of the physical object (1) and the image coordinate system (IMA) of the 3D image;
(b) computing a second coordinate transformation (^{INN1}T_{SUR}) between the coordinate system (SUR) of the surface fiducial markers (Fᵢ) attached to the physical object's outer side (2) and a coordinate system (INN₁) of an internal structure (I₁) inside the physical object (1);
(c) automatically extending the coordinate transformation (^{SUR}T_{IMA}) computed in step (a) by the coordinate transformation (^{INN1}T_{SUR}) computed in step (b) to create a coordinate transformation (^{INN1}T_{IMA}) between the image coordinate system (IMA) of the 3D image and the coordinate system (INN₁) of the internal structure (I₁).

31. The method according to claim 30, wherein the outer surface (2) is tracked by one of:
a. individually tracking the pose or position of the surface fiducial markers (Fᵢ), particularly by means of one of optical tracking, video-optical tracking, electromagnetic tracking, time-of-flight tracking;
b. tracking of the outer surface (2) by means of laser scanning or scanning with structured light.

32. The method according to claim 30, wherein additionally at least one adapter (A₁) is attached to the internal structure (Iᵢ) by a pose-trackable surgical robotic device (8) and wherein the at least one adapter (A₁) can be tracked relative to the surface fiducial markers (Fᵢ) by the processing unit (7) computing the coordinate transformations (^{SUR}T_{IMA} and ^{INN1}T_{SUR} and ^{INN1}T_{IMA}).
